(19) United States
(12) Patent Application Publication
(10) Pub. No.: US 2007/0282266 A1
(43) Pub. Date: Dec. 6, 2007

(54) BIFURCATED ENDOSCOPY CANNULA

(76) Inventor: Philip Davidson, St. Petersburg, FL (US)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 11/421,214

(22) Filed: May 31, 2006

(51) Int. Cl. A61M 5/178

(52) U.S. Cl. 604/164.01

(57) ABSTRACT

An endoscopy cannula system for accessing an internal operative site. The cannula access system includes a cannula having a tapered body and defining at least two access lumens extending therethrough. The two access lumens provide multiple access to the internal operative site from a single access location. The cannula further includes a fixation feature capable of securing the cannula relative to an access wound and to potentially providing retraction of overlying tissues proximal to the surgical site. The system also includes an obturator associated with each lumen. The obturators include a tapered distal tip which extends from the distal end of the cannula in an installed position. The tapered distal tips of the obturators provide the cannula with a generally continuously tapered profile to facilitate insertion through an access wound.

### FIELD

The present disclosure is generally directed at cannulas for providing endoscopic access to a work site.

### BACKGROUND

Endoscopy is a minimally invasive medical procedure that utilizes imaging equipment to view internal surfaces, features, or activities without requiring an extensive surgical opening which would permit direct observation by the clinician. For example an internal feature, such as the exterior surface of an internal organ, may be viewed by inserting a small viewing scope, such as an arthroscope or endoscope through a correspondingly small incision. The small size of the scope may allow a much smaller opening for viewing an internal feature than would be necessary for traditional, open direct observation by a clinician. Additionally, the scope may be an angled or a flexible device, allowing the scope to travel around or along other structures, internal organs, features, etc., before arriving at the site of interest. Achieving direct observation in such a situation could, therefore, require a major, highly invasive, surgical procedure.

In addition to merely viewing internal surfaces or features, endoscopy can be used for performing therapeutic procedures, etc. Generally, an endoscopic procedure may involve introducing an imaging device and a surgical instrument to an internal site of interest. The imaging device and the surgical instrument may be delivered through respective punctures or incisions. Once inside the patient, the instrument and imaging device must be triangulated, thereby spatially orienting the imaging device and the surgical instrument relative to one another. In part, triangulation places the working portion of the surgical instrument within the field of view of the imaging device so that the clinician can observe and appropriately manipulate the surgical instrument. During the procedure the imaging device and the surgical instrument must be continuously manipulated to provide the necessary visualization of the work site and of the surgical instrument to allow the procedure to be successfully carried out. The continual orientation and control of various devices and instruments introduced from different angles and locations can prove to be a very challenging task.

### BRIEF DESCRIPTION OF THE DRAWINGS

Features and advantages of the present invention are set forth by the description of embodiments consistent therewith, which description should be considered in conjunction with the accompanying drawings, wherein:

FIG. 1 is a perspective view of an embodiment of a cannula access system consistent with the present disclosure;

FIG. 2 is a front view of an embodiment of a cannula access system consistent with the present disclosure;

FIG. 3 is a cross-sectional view of an embodiment of a cannula access system consistent with the present disclosure;

FIG. 4 is an exploded view of a cannula consistent with the present disclosure;

FIG. 5 is a perspective view of an obturator which may suitably be used in connection with a cannula access system consistent with the present disclosure;

FIG. 6 is a representational view of a cannula anchored to a patient consistent with one embodiment of the present disclosure;

FIG. 7 is a diagrammatic view of a cannula and an embodiment of a fixation element consistent with the present disclosure;

FIG. 8 diagrammatically depicts the cannula of FIG. 7 with the fixation element in an installed position;

FIG. 9 depicts the cannula and fixation element of FIG. 7 employed to provide retraction;

FIG. 10 is another embodiment of an anchoring arrangement which may be used in connection with a cannula access system consistent with the present disclosure;

FIG. 11 shows the anchoring arrangement of FIG. 10 in a deployed configuration;

FIG. 12 shows another embodiment of an anchoring arrangement which may be used in connection with a cannula access system consistent with the present disclosure; and

FIG. 13 shows the anchoring arrangement of FIG. 12 in a deployed position.

### DESCRIPTION

The present disclosure relates to the field of endoscopy which, as used herein, may include, for example, laparoscopy, thoracoscopy, arthroscopy, etc. in which a “work site” is accessed from outside of a body. As used herein, “work site” may generally refer to a location of interest within the body, e.g., the location at which a therapeutic or diagnostic procedure is being carried out, i.e., an internal operative site. Endoscopy may involve the use of imaging equipment and the use of surgical instruments which may be inserted into a body, e.g. into a joint, the abdomen, etc., through one or more apertures made in the surface of the body. Endoscopic procedures may facilitate minimally invasive procedures.

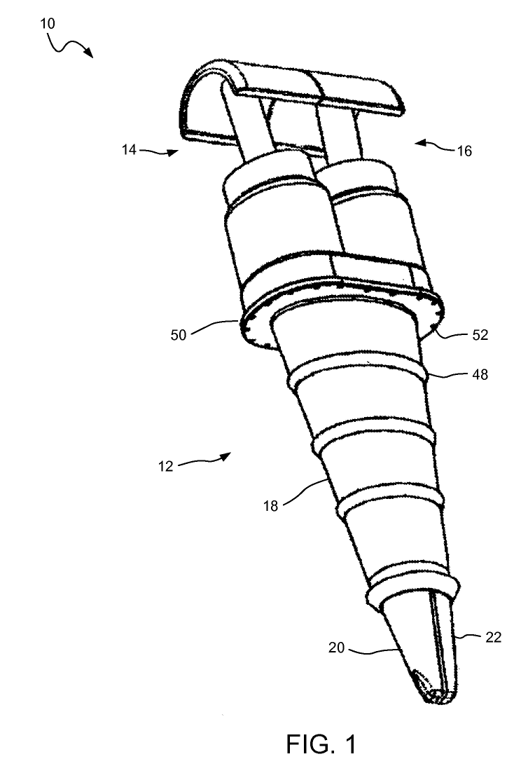

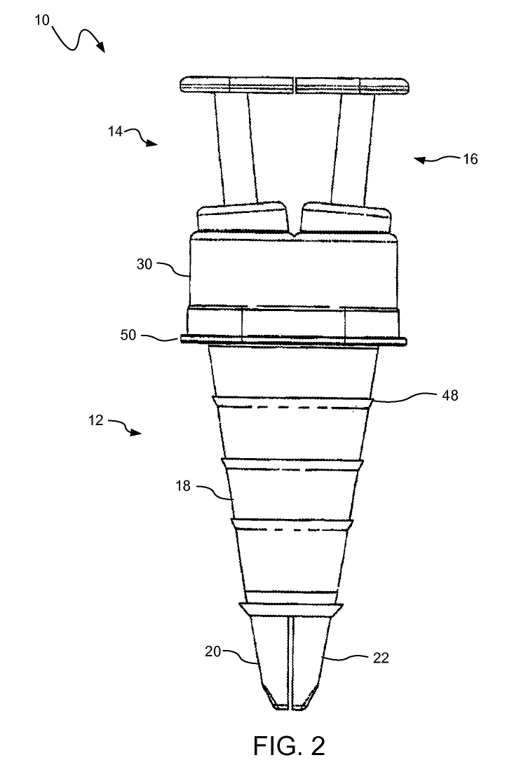

Referring to FIGS. 1 through 3, a cannula access system 10 is shown. The cannula access system 10 may generally include a multi-lumen cannula 12 and one or more obturators 14, 16 associated with each lumen of the cannula 12. The cannula 12 may have a generally tapered body 18 and the obturators 14, 16 may include tapered distal ends 20, 22. The converging tapers of the distal ends 20, 22 may cooperate to allow the obturators 14, 16 to generally extend the taper of the cannula 12 to provide a generally continuously tapered exterior.

The tapered shape of the cannula body 18, along with the tapered distal ends 20, 22 of the obturators 14, 16 may facilitate insertion of the cannula access system through a patient's skin, for example through an access wound, e.g., and access incision or puncture, in the patient's skin. When the obturators 14, 16 are assembled with the cannula 12, the tapered distal ends 20, 22 may function as trocars. The access incision or puncture may be formed in the patient's skin relative to an internal work site. The distal end of the cannula access system 10, that is the tapered distal ends 20, 22 of the obturators 14, 16, may be at least partially inserted into incision or puncture. Pressure may be applied to the cannula access system 10 forcing the cannula access system 10 through the incision or puncture toward the work site. In some procedures, the tapered exterior surface provided by the cannula 12 and the obturators 14, 16 may stretch, or otherwise enlarge, the incision or puncture allowing the cannula access system 10 to be positioned relative to the work site. The obturators 14, 16 may subsequently be removed from the cannula 12 to permit instruments, imaging equipment, etc., to access the work site through the cannula. Accordingly, multiple access pathways may be achieved using a single cannula inserted through a single portal, i.e., incision or puncture through the patient's skin.

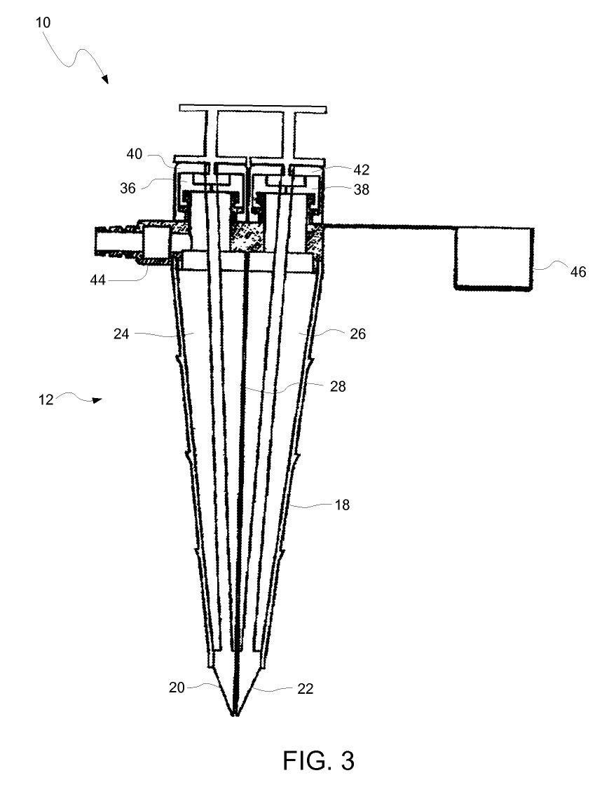

As shown with particular reference to the cross-sectional view of FIG. 3, the cannula 12 may include two or more lumens 24, 26, each of which may serve as a portal and provide access to a work site. Surgical and diagnostic instruments, as well as imaging or visualization equipment, such as cameras, fiber optic viewers, scopes, light sources, etc., may be received in the respective lumens 24, 26. In the foregoing manner, the multiple lumens 24, 26 may provide multiple access pathways to the work site from a single insertion point. In one embodiment, lumens may have converging axes. The individual lumens 24, 26 may be separated by a septum 28, e.g., in the form of an internal wall, such as may be integrally formed with the cannula 12, or may be a separately provided feature.

The single body of the cannula 12 including multiple lumens 24, 26 may provide a percutaneous access portal for instruments and imaging equipment. While the various instruments, imaging equipment, etc., may access a work site via separate lumens 24, 26, the single cannula 12 may provide a generally constant or fixed orientation or relationship between the instruments and imaging equipment disposed in separate lumens 24, 26, e.g., in the manner of yoking the equipment together. The orientation or relationship between the instruments, imaging equipment, etc., disposed in the separate lumens 24, 26 may assist in triangulation. For example, the common access point, achieved through a single access incision through the patient's skin, and the constant orientation or relationship provided by the lumens may eliminate the need for separately directing the instruments, imaging equipment, etc., within the patient to the work site. To this end, as mentioned above, in some embodiments, the lumens may have generally converging axes. In such an embodiment, instruments, imaging equipment, etc., introduced via the separate lumens may naturally tend toward a common point, e.g., at the work site.

In addition to assisting triangulation of the instruments, imaging equipment, etc., within the patient, the single access point provided by the multi-lumen cannula may facilitate approach to, as well as access and visibility of the work site. In some circumstances, there may be a limited number of approach paths to a desired work site. The multiple lumens 24, 26 of the cannula access system 10 may allow more than one instrument, imaging apparatus, etc., to access the work site along an optimum or at least acceptable approach path, without having to introduce each instrument or piece of imaging equipment along a separate approach path from a separate insertion point. For example, the cannula access system 10 may allow viewing access, as provided by a camera or other imaging device, and working access, e.g., by a surgical instrument, along a single approach path from a single insertion point through the patient's skin, e.g., from an especially advantageous insertion point and along an optimum approach path for accessing the particular work site.

Multiple access from a single insertion point, along a single approach path, as well as a constant or fixed orientation or relationship between various instruments and imaging devices may be especially advantageous when the work site is at least partially obstructed or difficult to approach. Various instruments and imaging equipment may be delivered to the work site via a single advantageous insertion point and approach path. Additionally, with a difficult work site, the problems associated with triangulation are simplified. Rather than trying to associate the imaging device and the operative instruments at the work site and from difficult insertion points, the instruments and operative equipment may be delivered to the work site having a known orientation or relationship.

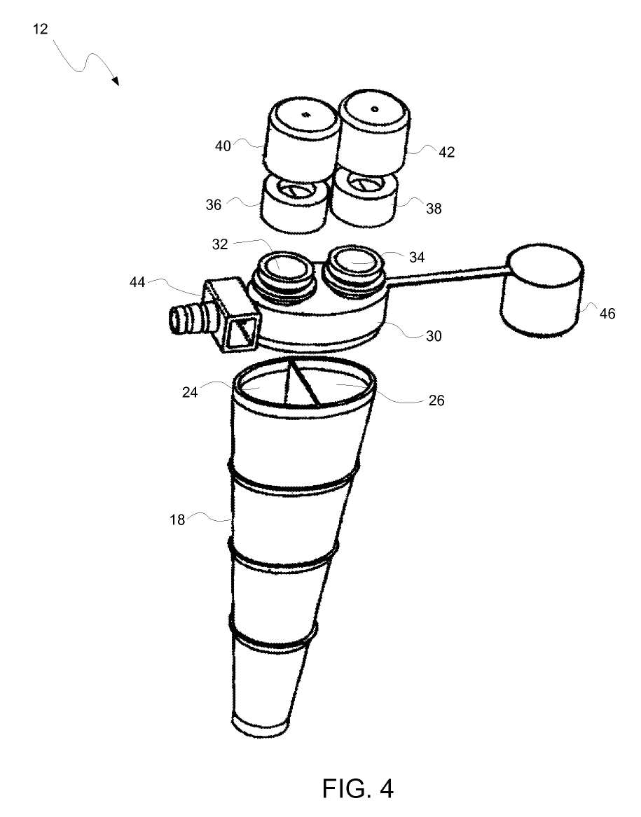

As best shown in FIGS. 3 and 4, the cannula 12 may include the tapered body 18, having a top piece 30 disposed at the proximal end thereof. The top piece 30 may define a first and second access opening 32, 34 associated with the respective lumens 24, 26 of the cannula 12. Respective inner 36, 38 and outer 40, 42 seals may be associated with the access openings 32, 34. The seals 36, 38, 40, 42 may resist, or limit, the ingress and egress of fluid, foreign matter, etc. through the cannula 12.

While resisting or limiting the ingress and egress of fluids or foreign matter through the cannula, the seals 36, 38, 40, 42 may also selectively provide access through the cannula 12, e.g., through elastic deformation or self-sealing punctures, slits, etc. For example, an instrument or piece of imaging equipment may be inserted through the inner and outer seals 36, 38, 40, 42 by opening a self sealing slit, expanding an aperture, etc. In various embodiments, the inner and outer seals 36, 38, 40, 42 may at least partially seal around an instrument or piece of imaging equipment inserted therethrough. Additionally, one or more of the inner and outer seals 36, 38, 40, 42 may at least partially re-seal the opening 32, 34 when the instrument or piece of imaging equipment is removed from the cannula 12.

The top piece 30 may include one or more irrigation features 44, which may allow the introduction of a fluid into at least one of the lumens 24. The irrigation feature 44 may provide valving, to allow the flow of an irrigating fluid, such as saline solution, etc., to be at least partially controlled at the cannula access system 10. The irrigation feature 44 may be disposed distally relative to the inner and outer seals 36, 40, which may restrict or limit the flow of fluid from the irrigation feature 40 and out through the proximal end of the cannula access system 10. A seal cap, e.g., 46, may be provided for at least one access opening 34. The seal cap 46 may sealing engage the outer seal 42, or other feature to restrict fluid or air outflow. In related embodiments, an irrigation feature may be configured to permit the infusion of a gas, such as air, though the irrigation portal. In further embodiments, a portal, such as the illustrated irrigation feature, may be configured to apply suction.

The cannula 12 may have a generally oval cross-sectional shape, e.g., elliptical, thereby providing increased lumen size. As such, the cannula 12 may have a tapered, generally elliptical shape. In other embodiments, the cannula 12 may have cross-sectional shapes other than elliptical, such as circular, polygonal, etc. Correspondingly, the top piece 30, seals 36, 38, 40, 42, etc., may similarly vary in configuration.

The various components of the cannula access system 10 may be produced from a variety of suitable materials. For example, the cannula 12 and top piece 30 may be any sterilizable material, such as a medical grade plastic, e.g., Hytrel® polyester elastomer, polycarbonate, acetal, etc. Similarly, the seals 35, 38, 40, 42 may be formed from a suitable elastomer, e.g., medical grade silicone, etc. The obturators 14, 16, or at least the distal tips 20, 22, may be formed from a relatively rigid material, such as polycarbonate, to facilitate forcing the cannula access system 10 into an access incision or puncture. The choice of materials herein should not be considered limiting on the scope of the disclosure, but rather merely as exemplary.

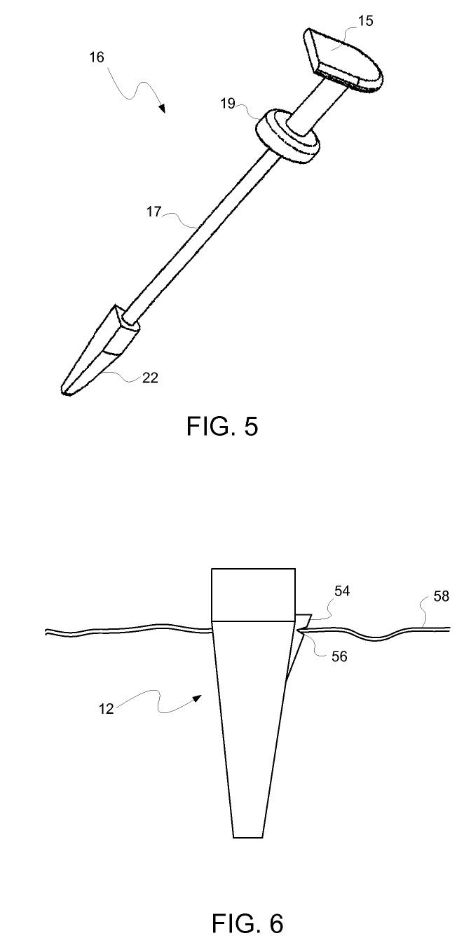

With additional reference to FIG. 5, each obturator, e.g., 16, may generally include a handle portion 15 connected to the tapered distal end 22 by a shaft portion 17, which may generally extend through the lumen 26 of the cannula 12 when the obturator 16 is assembled thereto. As shown in the various figures, the handle 15 may have a variety of configurations including curved, flat, etc. When the obturators 14, 16 are assembled with the cannula 12, the handles of the two or more obturators 14, 16 may be positioned to provide a generally continuous surface. The generally continuous surface provided by the handles of the obturators 14, 16 may facilitate pushing the cannula access system into an access incision or puncture to position the cannula 12 in the vicinity of the work site. The obturator 16 may include a flange 19 or other protrusion which may bear against a portion of the cannula 12, e.g., an upper portion of the outer seals 40, 42 to facilitate transmitting pressure applied to obturator handle 15 to the cannula 12.

The tapered profile of the cannula 12, as well as the conventional use of pressurized fluid for creating a pocket around the work site may tend to urge the cannula access system out of the access incision or puncture. Various features and arrangements may be used to anchor or secure the cannula 12 relative to the patient. The exemplary features and arrangements which follow should be understood as non-limiting examples of possible anchoring or securing arrangements.

The cannula access system 10 may be maintained in position relative to the patient's skin using a variety of fixation or securement features or techniques. As shown, the cannula may include a plurality of protrusions or ribs, e.g., 48, on the exterior of the cannula body 18. The ribs 48 may generally be arranged to engage the skin as the cannula 12 is inserted through an access incision or puncture. The ribs 48 may be distally tapered to facilitate insertion though the access incision or puncture, and may have a flat proximal extension to resist removal of the cannula 12.

The cannula 12 may include a suture feature, such as a suture flange 50. The suture flange 50 may be located exterior to the patient's skin when the cannula access system is installed for operative use. The suture flange 50 may include at least one suture opening 52, which may allow the cannula 12 to be sutured or stapled to the patient's skin surrounding the access incision or puncture. In a further embodiment, the suture flange may be formed from a penetrable material, which may allow sutures, staples, etc., to be inserted through the flange and into the underlying skin. In the foregoing manner, the cannula 12 may be anchored to the patient by direct suture or staple connection to skin at least partially surrounding the access incision or puncture. The suture flange 50 may be used in conjunction with retention features such as ribs 48, as shown, or may be used alone.

Another approach to anchoring or securing the cannula 12 relative to the patient is shown in FIG. 6. The cannula may be anchored or secured in the access incision or puncture using one or more fixation elements. As shown, an embodiment of a fixation element 54 may be generally configured as a wedge and may include a notch 56, which may receive skin 58 defining the access incision or puncture. The undercut provided by the notch 56 may accept the skin 58 defining the access incision or puncture, and may resist separation of the cannula 12 from the access incision or puncture, e.g., by necessitating expansion or stretching of the access incision or puncture. A single fixation element 54 may provide localized anchoring of the cannula 12, or a plurality of fixation elements spaced around the cannula may provide more continuous anchoring. In a related manner, the fixation element may extend around at least a portion, if not the entire, perimeter of the cannula.

The fixation element 54 may be provided as a separate component which may be assembled to the cannula or used in combination therewith. In one such embodiment, the fixation element 54 may be assembled to the cannula 12 before the cannula 12 is inserted into the access incision or puncture. The cannula 12 and the fixation element 54 may be inserted into the access incision or puncture until the fixation element 54 engages the skin 58 defining the access incision or puncture. In a related embodiment, the fixation element may be secondarily deployed, after or during insertion, below the skin, to effect security of the device and additionally to facilitate retraction of the skin and tissues overlying the surgical site. For example, the cannula 12 may be at least partially inserted into the access incision or puncture. The fixation element 54 may then be positioned at least partially between the cannula and the skin. The fixation element 54 may be maintained in position relative to the cannula 12 in a variety of ways, including frictional engagement, the use of adhesives, the use of cooperating mechanical features on the cannula and fixation element, etc. In a related embodiment, the fixation element may be attached to, integrally formed with, etc., the cannula. In such an embodiment, the cannula and fixation element may be installed in an access incision or puncture as a single component.

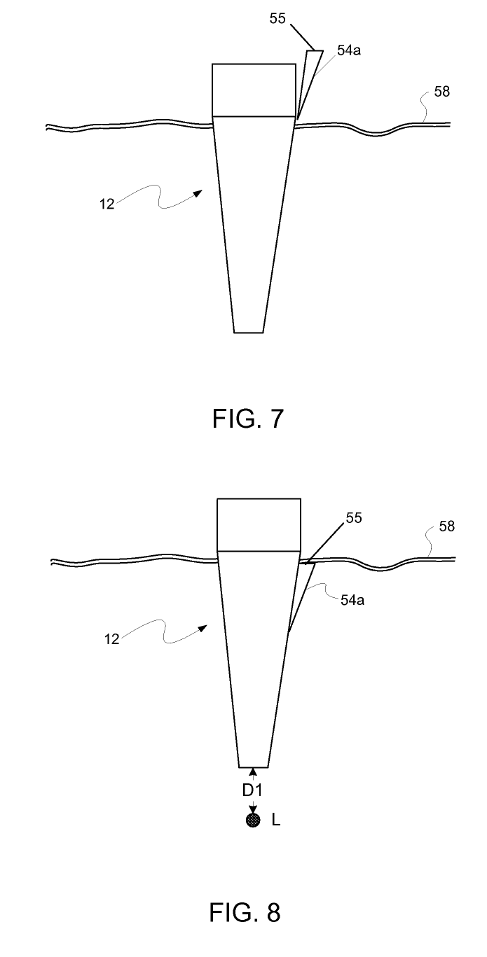

Referring to FIGS. 7 through 9, according to another embodiment, a fixation element 54a may be inserted below the skin 58. In a similar manner to the notch 56 of the fixation element 54 described with reference to FIG. 6, at least a portion of the proximal end 55 of the fixation element 54 may engage the underside of the skin 58 or other tissue surrounding the cannula 12. The fixation element may be retained relative to the cannula 12 in any suitable manner, including cooperating mechanical features, adhesive bonding, proximally extending features which may be disposed between the skin 58 and cannula 12, etc.

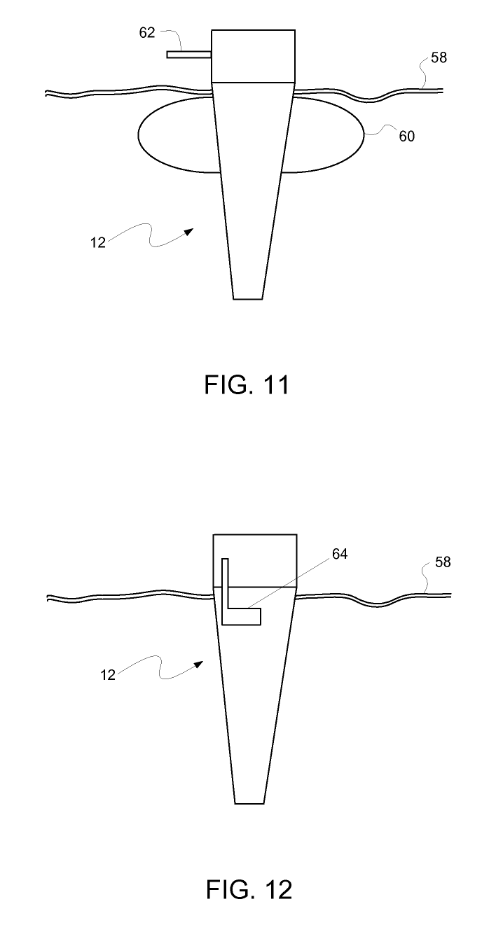

Turning next to FIGS. 10 and 11, yet anther arrangement is shown for anchoring or securing the cannula relative to the patient. As shown, the cannula 12 may include an inflatable or expandable feature 60 which may be deployed to resist separation of the cannula 12 from the patient. As shown, the cannula 12 may be inserted into the access incision or puncture with the inflatable or expandable feature 60 in a deflated or stowed configuration. When the cannula 12 has been positioned at least partially through the skin 58 defining the access incision or puncture, the inflatable or expandable feature 60 may be at least partially inflated or expanded beneath the skin 58. The enlarged cross-section created by the inflatable or expandable feature 60 may resist removal of the cannula 12 from the patient. The inflatable or expandable feature 60 may have a variety of configurations. For example, the feature 60 may be provided as an elastic membrane. The membrane may be inflated by introducing a fluid, e.g., saline, etc., between the membrane and the wall of the cannula, thereby inflating or expanding the feature 60. To this end, the cannula 12 may include an inflation port 62 fluidly coupled between the cannula wall and the membrane.

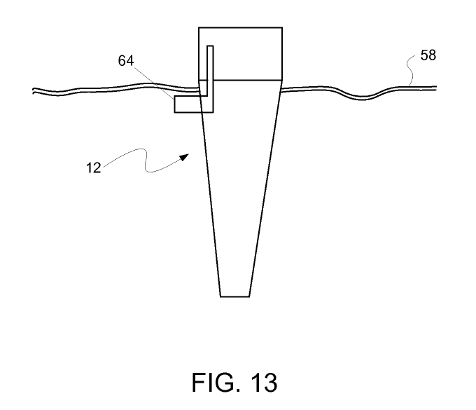

Still another arrangement for anchoring or securing a cannula to a patient is shown with reference to FIGS. 12 and 13. As shown, the cannula 12 may include a tab 64. The tab 64 may have a variety of configurations, such as a blade, paddle, retractor, etc. The tab 64 may be positioned in a stowed, or confluent, position, as shown in FIG. 12. With the tab 64 in the stowed position, the cannula 12 may be inserted into an access incision or puncture. The tab 64 may then be deployed at least partially beneath a patient's skin 58, as shown in FIG. 13. In the deployed position, the tab 64 may engage the patient's skin 58, or subcutaneous tissue and anchor the cannula 12 relative to the same. Additionally, the tab 64 may serve to retract the overlying skin, subcutaneous and any overlying tissue away from the underlying surgical site. As shown, the tab 64 may be generally pivotally movable relative to the cannula 12 between the stowed and deployed positions, although other embodiments may also be suitably employed. While only a single tab is depicted in FIGS. 12 and 13, it should be understood that an embodiment of a cannula may include a plurality of tabs.

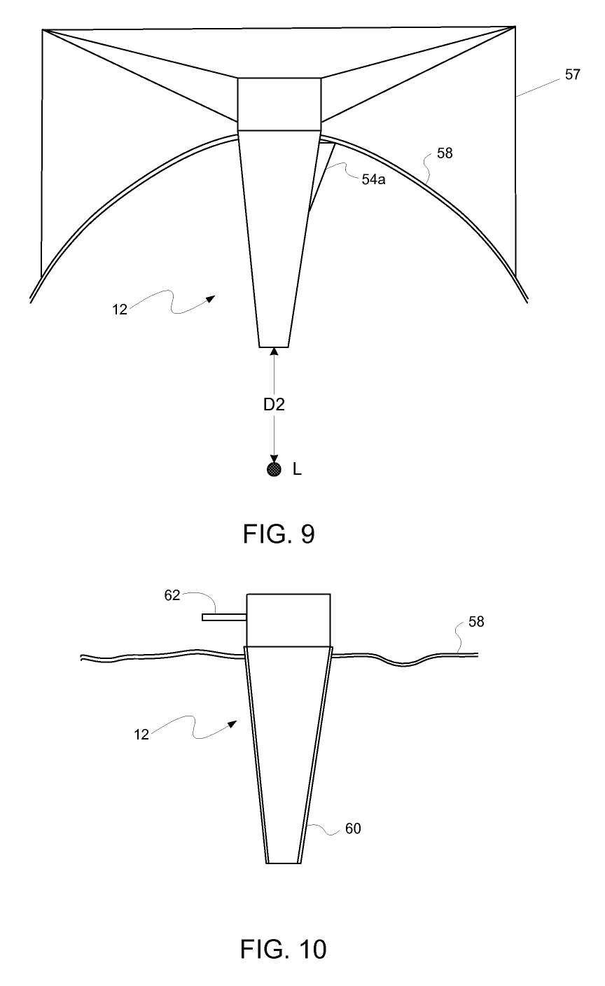

The task of viewing and maintaining the view of the surgical field of interest can be challenging during endoscopic procedures. The surrounding tissue can become swollen with fluid infused to facilitate the surgery, or overlying tissues may encroach upon the subjacent work area, leading to limitation of view and difficult access. In addition to anchoring or securing the cannula access system relative to the patient, the anchoring or securement features may allow the cannula to provide retraction relative to the work site. For example, as shown in FIGS. 8 and 9, once the cannula 12 is positioned relative to the work site L, e.g., a distance D1 from the work site L, the cannula 12 may be anchored or secured relative to the tissue, e.g., skin, overlying or adjacent to the work site, e.g., using a fixation element 54a. The cannula 12 may then be at least partially withdrawn proximally relative to the work site L, moving the distal end of the cannula 12 a distance D2 from the work site L, to create an operative pocket at the work site. While this aspect of the present disclosure has been illustrated with respect to the embodiment of FIGS. 8 and 9, it will be appreciated that similar use for retraction may be achieved with any suitable anchoring or securement feature used in connection with a cannula according to the present disclosure, including, but not limited to, any anchoring or securement feature depicted in any illustrated embodiment.

The cannula may be retracted by hand and may be maintained in an at least partially retracted position by hand. As shown in FIG. 9, the cannula 12 may also be retracted and/or maintained in an at least partially retracted position using a fixture 57. The fixture 57 may be supported against the patient, e.g. contacting the patient's skin 58, against an operating table, or other structure. Additionally, the fixture 57 may be configured to be coupled to the cannula 12. The cannula 12 may be retracted by hand or using the fixture 57. The cannula 12 may then be maintained in position, e.g., an at least partially retracted position, by the fixture 57. The fixture 57 depicted in FIG. 9 is a representative embodiment. A suitable fixture herein may be provided having many various configurations.

As the cannula is withdrawn relative to the work site, tissue surrounding the work site may be pulled away from the features of operative interest creating the operative pocket, and increasing access and visualization. In this manner, tissue may be pulled away from the features of operative interest by the cannula, rather than, or in addition to, being pushed away by the introduction of pressurized sterile water to the operative site. The occurrence of spilled or leaking water, and the associated mess and hazards associated with such an occurrence, at least in part, may be at least partially mitigated.

By way of example, in an arthroscopic procedure to repair a damaged ligament or tendon, an access incision or puncture may be made in the skin overlying the work site, i.e., the region of the damaged ligament. The distal ends of the obturators may be at least partially inserted into the access incision or puncture, and the cannula access system may be forced through the access incision or puncture to the region of the work site. The cannula may be anchored or secured to the skin surrounding the access incision or puncture, e.g., by suturing a flange of the cannula to the surrounding skin, etc. The cannula may then be at least partially withdrawn proximally from the work site, thereby at least partially drawing the skin, fat and subcutaneous tissues away from the work site and creating a pocket in the region of the damaged ligament. The pocket in the region of the damaged ligament may increase visualization and un-obstructed access to the damaged ligament. In this manner, retraction provided by the cannula may, at least in part, provide working room for carrying out the procedure. Of course, the cannula may be used in an analogous manner for various other endoscopic procedures.

In summary, according to one aspect of the present disclosure, a cannula is provided for endoscopic access to an internal operative site. The cannula may generally include a tapered body and at least a first and a second lumen extending through the body. The cannula may also include a fixation feature. The fixation feature may be capable of securing the body relative to tissue overlying at least a portion of the internal operative site.

According to another aspect, the present disclosure may provide a method for performing an endoscopic procedure. The method may include creating an access wound and inserting a cannula at least partially into the access wound. The method may also include securing the cannula relative to the access wound. The cannula may provide an access path to an internal operative site. Additionally, the method may include withdrawing the cannula at least partially away from the internal operative site. Withdrawing the cannula away from the internal operative site may retract tissue surrounding the internal operative site to create an operative pocket.

According to yet another aspect, the present disclosure may provide an endoscopy cannula access system. The cannula access system may include a cannula having a tapered body and at least two lumens extending through the body. The cannula may also include a fixation feature capable of securing the cannula relative to an access wound. The system may also include at least one obturator capable of being removably received in each of the at least two lumens. The obturators may each include a tapered distal end. At least a portion of the tapered distal end may extend from a distal end of the cannula in an installed position.

The embodiments described hereinabove are provided by way of illustration. It will be understood that the features and aspects of the various embodiments are susceptible to combination with one another. Furthermore, the features and aspects of the present disclosure are susceptible to modification and variation without departing materially from the spirit of the disclosure. Accordingly, the invention should not be limited by the disclosed embodiments, but only be the claims appended hereto.

What is claimed is:

1. A cannula for endoscopic access to an internal operative site comprising:
a tapered body and at least a first and second lumen extending therethrough; and
a fixation feature capable of securing said body relative to tissue overlying at least a portion of said internal operative site.

2. A cannula according to claim 1, wherein said fixation feature comprises at least one rib extending around at least a portion of an exterior of said body, said rib capable of engaging tissue surrounding said cannula.

3. A cannula according to claim 1, wherein said fixation feature comprises a suture feature comprising at least one opening capable of receiving a suture for coupling said cannula to surrounding tissue.

4. A cannula according to claim 1, wherein said fixation feature comprises an undercut capable of receiving at least a portion of said tissue.

5. A cannula according to claim 1, further comprising at least one seal disposed relative to at least one of said first and second lumens, said at least one seal capable of limiting ingress and egress of a fluid through said cannula.

6. A cannula according to claim 1, further comprising at least a first and second obturator capable of being removably disposed in said first and second lumens.

7. A cannula according to claim 6, wherein said first and second obturators comprise a generally tapered distal end extending from said body in an installed position.

8. A cannula according to claim 1, wherein said first and second lumens comprise converging axes.

9. A cannula according to claim 1, wherein said tapered body comprises a generally oval cross-sectional geometry

10. A method of performing an endoscopic procedure comprising:
creating an access wound;
inserting a cannula at least partially into said access wound;
securing said cannula relative to said access wound, said cannula providing an access path to an internal operative site; and
withdrawing said cannula at least partially away from said internal operative site, said cannula retracting tissue surrounding said internal operative site away from said internal operative site to create an operative pocket.

11. A method according to claim 10, wherein securing said cannula relative to said access wound comprises suturing at least a portion of said cannula to skin adjacent to said access wound.

12. A method according to claim 10, wherein securing said cannula relative to said access wound comprises engaging tissue surrounding said access wound.

13. A method according to claim 12, wherein engaging tissue surround said access wound comprises deploying at least a portion of a fixation feature below at least a portion of said tissue.

14. A method according to claim 10, wherein said cannula further comprises at least on obturator comprising a tapered distal tip, and inserting said cannula comprises disposing said tapered distal tip at least partially in said access wound and forcing said cannula at least partially through said access wound.

15. An endoscopy cannula access system comprising:
a cannula comprising an tapered body and at least two lumens extending therethrough, and a fixation feature capable of securing said cannula relative to an access wound; and
at least one obturator capable of being removably received in each of said lumens, said obturators each comprising a tapered distal end, at least a portion of said tapered distal end extending from a distal end of said cannula in an installed position.

16. The system according to claim 15, wherein said obturators each comprise a proximal handle capable of extending from a proximal end of said cannula in said installed position, said handles capable of transmitting an insertion force to said cannula for inserting said cannula into an access wound.

17. The system according to claim 15, wherein said fixation feature comprises a suture feature comprising at least on suture opening for suturing said cannula to tissue at least partially surrounding said access wound.

18. The system according to claim 15, wherein said fixation feature comprises an undercut capable of engaging tissue surrounding said access wound.

19. The system according to claim 15, wherein said fixation feature comprises at least one rib outwardly projecting from said body.

20. The system according to claim 15, wherein said cannula further comprises at least one seal associated with each lumen, said seal capable of limiting ingress and egress of fluid via said cannula.
